(19) United States
(12) Patent Application Publication
(10) Pub. No.: US 2026/0174675 A1
(43) Pub. Date: Jun. 25, 2026

(54) INJECTABLE AQUEOUS-BASED LEUCOVORIN FORMULATION AND PREPARATION METHOD THEREOF

(71) Applicant: Riconpharma LLC, Denville, NJ (US)

(72) Inventors: Vinay Sudhindra Raichur, Hyderabad (IN); Vijaya Kumar Thota, Hyderabad (IN); SVB Janardhan Garikipati, Hyderabad (IN); Praveen Reddy Billa, Denville, NJ (US); Mukteeshwar Gande, Denville, NJ (US)

(73) Assignee: Riconpharma LLC, Denville, NJ (US)

(21) Appl. No.: 18/991,888

(22) Filed: Dec. 23, 2024

(51) Int. Cl. A61K 9/00; A61K 9/08; A61K 31/519; A61K 47/02; A61K 47/18; A61K 47/40

(52) U.S. Cl. CPC A61K 9/0019; A61K 9/08; A61K 31/519; A61K 47/02; A61K 47/18; A61K 47/40

(57) ABSTRACT

The present invention relates to a stable aqueous formulation of Leucovorin calcium designed for parenteral administration. This formulation utilizes substituted cyclodextrins, specifically Betadex sulfobutyl ether cyclodextrin (SBE-β-CD) or Hydroxypropyl Beta Cyclodextrin (HP-β-CD), as solubilizers and stabilizers to enhance the drug's aqueous solubility. The inclusion of Tromethamine serves as a buffering agent, maintaining optimal pH levels and ensuring the chemical stability of Leucovorin over an extended shelf life. Importantly, the formulation effectively prevents drug crystallization and particulate matter formation, addressing major stability challenges associated with existing products. The formulation's resilience to freeze-thaw cycles confirm its physical and chemical stability under stress conditions. These advancements not only improve the safety and efficacy of Leucovorin calcium for intravenous administration but also facilitate compliance with regulatory standards, providing a reliable therapeutic option for patients.

### FIELD OF THE INVENTION

This invention generally relates to pharmaceutical formulations and more specifically, a stable aqueous injectable formulation that improve the physical and chemical stability of Leucovorin calcium by enhancing its solubility and preventing drug crystallization.

### BACKGROUND OF THE INVENTION

Leucovorin, a chemically reduced derivative of folic acid, is commonly marketed as Leucovorin calcium, its salt form. The currently available liquid injection of Leucovorin is formulated as a 10 mg/mL solution, primarily produced by Fresenius Kabi. Historically, Bedford Laboratories (now Hikma) also offered a similar 10 mg/mL Leucovorin calcium injection. However, multiple product recalls have been reported due to the presence of particulate matter in these injections, as highlighted by FDA MedWatch. Such recalls underscore significant challenges in the physical stability of Leucovorin calcium formulations, particularly concerning the prevention of drug crystallization and particulate formation.

The typical formulation of Leucovorin calcium liquid injection consists of 10 mg/mL of the leucovorin base, 8 mg/mL of sodium chloride, and sodium hydroxide and/or hydrochloric acid for pH adjustment within a range of 6.5 to 8.5. Despite this carefully designed composition, the product has encountered substantial difficulties related to the low solubility of Leucovorin. This solubility issue often leads to crystallization, which subsequently results in the formation of particulate matter, rendering the product unsuitable for intravenous administration.

Currently, there is only one commercial liquid formulation of Leucovorin available, which also features a concentration of 10 mg/mL and includes 8 mg/mL sodium chloride. This formulation is intended for storage at 2-8° C. The inherent solubility challenge and risk of particulate matter formation emphasizes the need for a more stable formulation that can maintain both physical and chemical integrity, particularly under various storage conditions, to ensure safety and efficacy for patients requiring intravenous treatment.

Leucovorin formulations disclosed in the prior art exhibits several disadvantages that limit the effectiveness of the formulation. Many of these formulations combine Leucovorin with other active ingredients or stabilizers, complicating their therapeutic use. For instance, the inclusion of 5-fluorouracil alongside Leucovorin may not be suitable for patients who require Leucovorin as a standalone treatment. Furthermore, while some formulations report short-term stability, they lack comprehensive long-term stability data essential for ensuring product safety and efficacy over time.

Certain formulations contain lower concentrations of Leucovorin (e.g., 5 mg/mL), which fall below the saturation solubility, limiting therapeutic effectiveness. Additionally, many have not undergone rigorous stress testing, which are crucial for understanding how the product will perform in real-world conditions. Moreover, some formulations utilize excipients, like sodium gluconate, at concentrations exceeding the limits set by the Inactive Ingredient Guide (IIG), raising safety concerns. Other formulations include excipients not listed in the IIG, which could complicate regulatory approval. Finally, although some prior art attempts to address crystallization, they often do so inadequately, failing to employ solubilizers or stabilizers designed specifically for this issue. Collectively, these disadvantages underscore the urgent need for innovative formulations that effectively enhance the stability of Leucovorin while ensuring compliance with safety standards for intravenous administration.

Thus, there remains a need for a more stable formulation of Leucovorin calcium that not only enhances its solubility but also addresses the long-term physical and chemical stability of the formulation.

### BRIEF SUMMARY OF THE INVENTION

The first aspect of the present invention provides an injectable aqueous-based Leucovorin formulation. The formulation comprises (i) Leucovorin calcium at a concentration of 10 to 20 mg/mL, (ii) sodium chloride as a co-solubilizer at a concentration of 4 to 12 mg/mL (iii) Betadex Sulfobutyl Ether Sodium (SBE-β-CD) or Hydroxy Propyl Beta Cyclodextrin (HP-β-CD) as a stabilizer and a solubilizer at a concentration of about 155 mg/mL, (iv) Tromethamine as a buffering agent at a concentration of about 8 mg/mL, and Water for Injection (WFI) q.s. to 1 ml. pH of the formulation is adjusted preferably to a range of 6.5 to 8.5, and more preferably to a range of 8.0 to 8.5 using hydrochloric acid (HCl) or sodium hydroxide (NaOH).

In an embodiment, the formulation comprises sodium chloride as the co-solubilizer at a concentration of 4 mg/mL.

The second aspect of the present invention provides a method for preparing an injectable aqueous-based Leucovorin formulation. The method comprises (i) dissolving sodium chloride in Water for Injection (WFI) at a concentration of 4 to 12 mg/mL to obtain a first solution; (ii) adding Tromethamine to the first solution at a concentration of about 8 mg/mL to obtain a second solution; (iii) adding Betadex Sulfobutyl Ether Sodium (SBE-β-CD) or Hydroxypropyl Beta Cyclodextrin (HP-β-CD) to the second solution at a concentration of about 155 mg/mL to obtain a third solution; (iv) adjusting pH of the third solution preferably to a range of 6.5 to 8.5, and more preferably to a range of 8.0 to 8.5 using hydrochloric acid (HCl) or sodium hydroxide (NaOH); (v) adding Leucovorin calcium at a concentration of 10 to 20 mg/mL to the third solution by continuous stirring to obtain a fourth solution, the third solution is stirred until the Leucovorin calcium is fully dissolved; and (vi) adjusting final volume of the fourth solution by adding the Water for Injection (WFI) to obtain the injectable aqueous-based Leucovorin formulation.

In an embodiment, the method comprises adding sodium chloride as a co-solubilizer at a concentration of 4 mg/mL.

In another embodiment, the method comprises (i) filtering the formulation through a 0.22 μm sterile filter; and (ii) aseptically transferring the sterile formulation into containers.

The present invention offers several significant advantages in the formulation of Leucovorin calcium for parenteral administration. By utilizing substituted cyclodextrins, such as Betadex Sulfobutyl ether cyclodextrin (SBE-β-CD) or Hydroxypropyl Beta Cyclodextrin (HP-β-CD), the formulation achieves enhanced aqueous solubility, allowing for higher concentrations of the active ingredient and improved therapeutic efficacy. The solubility enhancement provides drug stabilization, greatly reducing the tendency to agglomerate or precipitate, as intramolecular interactions of Leucovorin are inhibited. This minimizes the formation of crystal seeds due to molecular association, which is a primary cause of physical instability in Leucovorin and its salts. Additionally, the inclusion of Tromethamine as a buffering agent ensures optimal pH maintenance, addressing pH drift which is a major cause of chemical instability for folate analogues thereby preserving the chemical stability of Leucovorin over an extended shelf life. The formulation effectively prevents drug crystallization and the formation of particulate matter, addressing critical stability challenges faced by existing products and significantly reducing the risk of product recalls. Moreover, it demonstrates resilience under stress conditions, such as freeze-thaw cycles, confirming its physical and chemical stability even during adverse storage conditions. These features not only enhance patient safety by ensuring the product remains suitable for intravenous administration but also contribute to a longer shelf life and regulatory compliance, facilitating easier market access. Overall, this invention represents a significant advancement in the formulation of Leucovorin calcium, improving both its stability and therapeutic utility.

### BRIEF DESCRIPTION OF THE DRAWINGS

A clear understanding of the key features of the invention summarized above may be had by reference to the appended drawings, which illustrate the method and system of the invention, although it will be understood that such drawings depict preferred embodiments of the invention and, therefore, are not to be considered as limiting its scope with regard to other embodiments which the invention is capable of contemplating. Accordingly:

FIG. 1—FIG. 1 illustrates a method for preparing an injectable aqueous-based Leucovorin formulation according to various embodiments of the present invention.

Like reference numerals refer to like parts throughout the several views of the drawings.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

Nevertheless, the specification and claims should be read with the understanding that such combinations are entirely within the scope of the invention and the claims. An injectable aqueous-based leucovorin formulation and preparation method thereof is discussed herein. In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the FIGURES or description below. The present invention will now be described by referencing the appended FIGURES representing preferred embodiments.

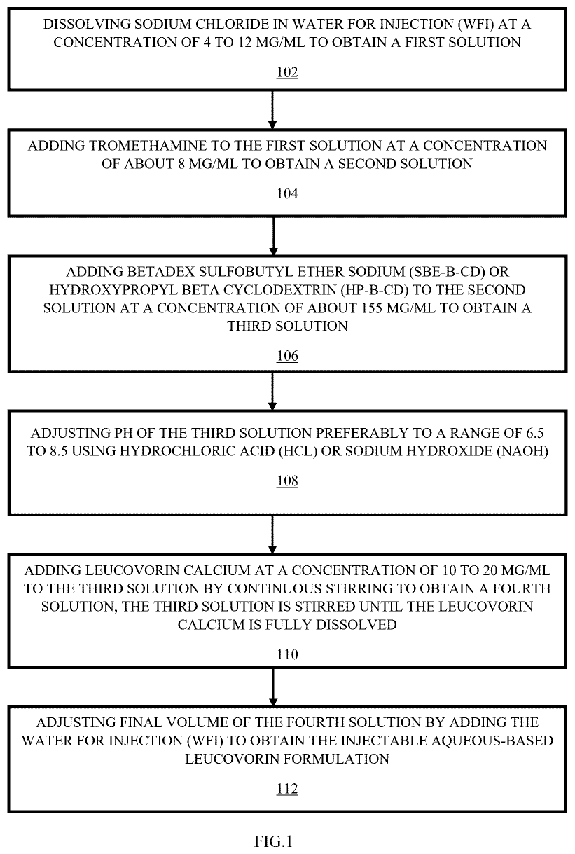

FIG. 1 illustrates a method for preparing an injectable aqueous-based Leucovorin formulation according to various embodiments of the present invention.

At step 102, the method includes dissolving sodium chloride in Water for Injection (WFI) at a concentration of 4 to 12 mg/mL to obtain a first solution;

At step 104, the method includes adding Tromethamine at a concentration of about 8 mg/mL to the first solution to obtain a second solution;

At step 106, the method includes adding Betadex Sulfobutyl Ether Sodium (SBE-β-CD) or Hydroxypropyl Beta Cyclodextrin (HP-β-CD) to the second solution at a concentration of about 155 mg/mL to obtain a third solution;

At step 108, the method includes adjusting pH of the third solution preferably to a range of 6.5 to 8.5, and more preferably to a range of 8.0 to 8.5 using hydrochloric acid (HCl) or sodium hydroxide (NaOH).

At step 110, the method includes adding Leucovorin calcium at a concentration of 10 to 20 mg/mL to the third solution by continuous stirring to obtain a fourth solution, the third solution is stirred until the Leucovorin calcium is fully dissolved; and

At step 112, the method includes adjusting the final volume of the fourth solution by adding the Water for Injection (WFI) to achieve a total volume of 1 mL to obtain the injectable aqueous-based Leucovorin formulation.

In an embodiment, the WFI is collected and maintained at a controlled temperature between 20° C. and 25° C. throughout the process to ensure the stability and solubility of leucovorin in the aqueous solution. After the collection of WFI, the next step involves purging the solution with filtered nitrogen gas. This reduces and controls the dissolved oxygen content in the aqueous solution, preventing oxidation and degradation of leucovorin. Nitrogen is introduced until the target dissolved oxygen content is achieved, ensuring the dissolved oxygen level falls within a predefined range that is optimal for the stability of the leucovorin formulation.

TABLE 1
presents the formulation details for Leucovorin injection in two
variations: RS (Reference Standard), and proposed formulation.

Composition, mg/mL

| Ingredient | RS | Proposed Formulation |
|---|---|---|
| Leucovorin | 10 | 10 to 20 mg |
| Betadex Sulfobutyl | — | 155 mg |
| Ether Sodium | | |
| (SBE-β-CD) | | |
| Tromethamine | — | 8 mg |
| NaCl | 8 | 4 to 12 mg |
| NaOH | QS for pH | QS for pH adjustment |
| | adjustment | to 8.0 to 8.5 |
| HCl | QS for pH | QS for pH adjustment |
| | adjustment | to 8.0 to 8.5 |
| WFI | QS to 1 mL | QS to 1 mL |

Each formulation contains at least 10 mg/mL of Leucovorin as the active ingredient. RS formulation includes 8 mg/mL of NaCl (sodium chloride), while proposed formulation has NaCl in the range of 4-12 mg/mL and more preferably 4 mg/mL. The proposed formulation has Tromethamine of about 8 mg/mL and more preferably 7 mg/mL as a buffering agent, which is absent in RS formulations. Additionally, proposed formulation includes about 155 mg/mL of Betadex Sulfobutyl Ether Sodium (SBE-β-CD), a solubilizer, which is not present in RS. The formulations use sodium hydroxide (NaOH) and hydrochloric acid (HCl) for pH adjustment, with proposed formulation specifically targeting a pH range of 8.0 to 8.5. In all formulations, Water for Injection (WFI) is added to bring the total volume to 1 mL. The proposed product presents the claimed formulation designed for enhanced solubility and stability. The following examples further describe certain specific aspects and embodiments of the present invention and demonstrate the practice and advantages thereof. It is to be understood that the examples are given by way of illustration only and are not intended to limit the scope of the invention in any manner.

### Example 1: Leucovorin Injection Formulation

| S. No | Ingredients | Qty/mL |
|---|---|---|
| 1 | Leucovorin calcium | 10 mg |
| 2 | Betadex Sulfobutyl | 50 mg |
| | Ether Sodium | |
| 3 | Sodium chloride | 4 mg |
| 4 | Hydrochloric acid | Q.S to adjust pH |
| 5 | Sodium hydroxide | Q.S to adjust pH |
| 6 | Water for Injection | Q.S to mL |

The Leucovorin injection formulation of example 1 was prepared by adding sodium chloride (4 mg) to a compounding vessel containing Water for Injection (WFI). Betadex Sulfobutyl Ether Sodium (50 mg) was added and stirred till a uniform solution was formed. pH was adjusted followed by addition of Leucovorin calcium (10 mg) and water and stirred till a uniform solution was obtained. The solution was filtered, followed by stoppering, and sealing of the vials.

Leucovorin calcium formulation prepared according to example 1 was tested for stability under long term condition for period of 6 month at 2-8° C. and under accelerated condition for a period of 6 months at 25° C./60% RH. The stability data is summarized in Table 2 and Table 3.

TABLE 2
presents stability data at 2-8° C. long term condition of
proposed formulation in example 1.
Stability study data of Leucovorin Calcium Injection USP 10 mg/mL

| Test | | 1 | 2 | 3 | 6 |
|---|---|---|---|---|---|
| parameters | Initial | month | month | month | month |
| Description | A clear, colourless to yellow colour solution | | | | |
| pH | 7.73 | 7.77 | 7.76 | 7.81 | 7.79 |
| Osmolality | 385 | 383 | 380 | 382 | 383 |
| Assay | 101.8 | 103.2 | 103.9 | 103.3 | 104.3 |

Related substance

| | | | | | |
|---|---|---|---|---|---|
| Impurity E | 0.044 | 0.044 | 0.047 | 0.049 | 0.044 |
| Impurity A | 0.052 | 0.078 | 0.097 | 0.097 | 0.273 |
| Impurity F | 0.065 | 0.065 | 0.074 | 0.080 | 0.226 |
| Impurity B | 0.000 | 0.000 | 0.000 | 0.010 | 0.000 |
| Impurity G | 0.000 | 0.000 | 0.015 | 0.006 | 0.000 |
| Impurity D | 0.000 | 0.000 | 0.006 | 0.014 | 0.000 |
| Impurity C | 0.000 | 0.000 | 0.005 | 0.000 | 0.011 |
| THFA | 0.000 | 0.000 | 0.004 | 0.000 | 0.000 |
| Single max | 0.05 | 0.06 | 0.032 | 0.042 | 0.09 |
| Total | 0.21 | 0.25 | 0.28 | 0.30 | 0.64 |

TABLE 3
presents stability data at 25° C./60% RH accelerated condition of
proposed formulation in example 1.
Stability study data of Leucovorin Calcium Injection USP 10 mg/mL

| Test | | 1 | 2 | 3 | 6 |
|---|---|---|---|---|---|
| parameters | Initial | month | month | month | month |
| Description | A clear, colourless to yellow colour solution | | | | |
| pH | 7.73 | 7.77 | 7.63 | 7.79 | 7.68 |
| Osmolality | 385 | 104.3 | 381 | 384 | 380 |
| Assay | 101.8 | 103.7 | 100.9 | 100.8 | 99.7 |

Related substance

| | | | | | |
|---|---|---|---|---|---|
| Impurity E | 0.044 | 0.044 | 0.046 | 0.069 | 0.044 |
| Impurity A | 0.052 | 0.260 | 0.643 | 0.758 | 1.857 |
| Impurity F | 0.065 | 0.323 | 0.575 | 0.471 | 1.323 |
| Impurity B | 0.000 | 0.000 | 0.000 | 0.000 | 0.022 |
| Impurity G | 0.000 | 0.000 | 0.007 | 0.000 | 0.143 |
| Impurity D | 0.000 | 0.000 | 0.013 | 0.013 | 0.025 |
| Impurity C | 0.000 | 0.000 | 0.005 | 0.000 | 0.000 |
| THFA | 0.000 | 0.000 | 0.001 | 0.000 | 0.000 |
| Single max | 0.05 | 0.06 | 0.173 | 0.267 | 0.55 |
| Total | 0.21 | 0.69 | 1.46 | 1.58 | 3.96 |

The formulation is subjected to freeze thaw study at −20° C. to 2-8° C. for physical stability with 24 hours hold at each temperature, and the study was performed on multiple cycles (>10 cycles). Invention was found to be stable with no significant signs of crystallization after frequent cycles. The data confirms the finding that the use of suitable solubilizer in suitable proportions provide best results.

### Example 2: Leucovorin Injection Formulation

| S. No | Ingredients | Qty/mL |
|---|---|---|
| 1 | Leucovorin calcium | 20 mg |
| 2 | Betadex Sulfobutyl | 155 mg |
| | Ether Sodium | |
| 3 | Sodium chloride | 4 mg |
| 4 | Hydrochloric acid | Q.S to adjust pH |
| 5 | Sodium hydroxide | Q.S to adjust pH |
| 6 | Water for Injection | Q.S to mL |

Leucovorin Injection formulation of example 2 was prepared by adding sodium chloride (4 mg) to the compounding vessel containing Water for Injection (WFI). Betadex Sulfobutyl Ether Sodium (155 mg) was added and stirred till a uniform solution was formed. pH was adjusted followed by addition of Leucovorin calcium (20 mg) stirred till a uniform solution was obtained. Volume was made up to 100% of batch size. The solution was filtered, followed by stoppering, and sealing of the vials.

Leucovorin calcium formulation prepared according to the example 2 was tested for stability under long term condition for period of 3 month at 2-8° C. for long term stability. The stability data is summarized in Table 4.

TABLE 4
presents stability data at 2-8° C. long term condition of
proposed formulation in example 2.
Stability study data of Leucovorin Calcium Injection USP 20 mg/mL

| Test | | 1 | 3 |
|---|---|---|---|
| parameters | Initial | month | months |
| Description | A clear, colourless to yellow colour solution | | |
| pH | 7.8 | 7.63 | 7.66 |
| Osmolality | 507 | 495 | 506 |
| Assay | 103.1 | 102.8 | 104.7 |

Related substance

| | | | |
|---|---|---|---|
| Impurity E | 0.044 | 0.044 | 0.044 |
| Impurity A | 0.026 | 0.052 | 0.078 |
| Impurity F | 0.032 | 0.097 | 0.065 |
| Impurity B | 0.000 | 0.000 | 0.000 |
| Impurity G | 0.000 | 0.000 | 0.000 |
| Impurity D | 0.075 | 0.075 | 0.050 |
| Impurity C | 0.000 | 0.000 | 0.011 |
| THFA | 0.000 | 0.000 | 0.000 |
| Single max | 0.06 | 0.05 | 0.05 |
| Total | 0.24 | 0.32 | 0.30 |

The formulation is subjected to freeze thaw study at −20° C. to 2-8° C. for physical stability with 24 hours hold at each temperature, and the study was performed on multiple cycles (>10 cycles). The formulation was found to be stable with no significant signs of crystallization after frequent cycles. The data confirms the finding that the use of suitable solubilizer in suitable proportions provide best results.

The injectable aqueous-based formulation of Leucovorin of the present invention offers numerous critical applications in medical practice, particularly in oncology and hematology. Primarily, it serves to enhance the efficacy of chemotherapy agents by rescuing normal cells from toxicity. This stable formulation is also effective in treating folate deficiency and megaloblastic anemia, providing a reliable option for patients unable to take oral supplements. Additionally, it plays a vital role in mitigating toxicity from antifolate drugs during overdose situations, facilitating patient recovery. The incorporation of substituted cyclodextrins enhances the solubility and bioavailability of Leucovorin, allowing for higher concentrations to be delivered safely. Its long-term stability, demonstrated through resistance to crystallization and performance under freeze-thaw conditions, ensures that the medication remains effective throughout its shelf life. Furthermore, adherence to safety standards and easy intravenous administration make this formulation a versatile and trustworthy choice for healthcare providers, ultimately improving patient outcomes. SBE-β-CD is known for its beneficial effect in enhancing the aqueous solubility and chemical stability of drugs by forming inclusion complexes. The formed complex will undergo rapid dissociation upon getting exposed to blood circulation, thereby minimizing accumulation of drug substance in body thus eliminating the adverse effects. Further, SBE-β-CD upon intravenous administration undergoes renal filtration and gets eliminated via urine which indicates that it has no or less nephrotoxicity.

While the present invention has been described in terms of particular embodiments and applications, in both summarized and detailed forms, it is not intended that these descriptions in any way limit its scope to any such embodiments and applications. It will be understood that many substitutions, changes and variations in the described embodiments, applications and details of the method and system illustrated herein and of their operation can be made by those skilled in the art without departing from the spirit of this invention.

What is claimed is:

1. An injectable aqueous-based Leucovorin formulation, comprising:
Leucovorin calcium at a concentration of 10 to 20 mg/mL;
Betadex Sulfobutyl Ether Sodium (SBE-β-CD) or Hydroxy Propyl Beta Cyclodextrin (HP-β-CD) as a stabilizer and a solubilizer at a concentration of about 155 mg/mL;
Sodium chloride as a co-solubilizer at a concentration of 4-12 mg/mL;
Tromethamine as a buffering agent at a concentration of about 8 mg/mL; and
Water for Injection (WFI) q.s. to 1 mL, wherein pH of the formulation is adjusted preferably to a range of 6.5 to 8.5 using hydrochloric acid (HCl) or sodium hydroxide (NaOH).

2. The formulation according to claim 1, wherein the formulation comprises sodium chloride as the co-solubilizer at a concentration of 4 mg/mL.

3. A method for preparing an injectable aqueous-based Leucovorin formulation, wherein the method comprises:
dissolving sodium chloride in Water for Injection (WFI) at a concentration of 4 to 12 mg/mL to obtain a first solution;
adding Tromethamine to the first solution at a concentration of about 8 mg/mL to obtain a second solution;
adding Betadex Sulfobutyl Ether Sodium (SBE-β-CD) or Hydroxypropyl Beta Cyclodextrin (HP-β-CD) to the second solution at a concentration of about 155 mg/mL to obtain a third solution;
adjusting pH of the third solution preferably to a range of 6.5 to 8.5 using hydrochloric acid (HCl) or sodium hydroxide (NaOH);
adding Leucovorin calcium at a concentration of 10 to 20 mg/mL to the third solution by continuous stirring to obtain a fourth solution, wherein the third solution is stirred until the Leucovorin calcium is fully dissolved; and
adjusting final volume of the fourth solution by adding the Water for Injection (WFI) to obtain the injectable aqueous-based Leucovorin formulation.

4. The method according to claim 3, wherein the formulation comprises sodium chloride as the co-solubilizer at a concentration of 4 mg/mL.

5. The method according to claim 3, wherein the method comprises (i) filtering the formulation through a 0.22 μm sterile filter; and (ii) aseptically transferring the sterile formulation into containers.
